(19) United States
(12) Patent Application Publication
(10) Pub. No.: US 2026/0232553 A1
(43) Pub. Date: Aug. 13, 2026

(54) MULTI-FUNCTIONAL ACTIVE COMPLEX FOR RESTORATION OF SKIN BARRIER AND SEBUM REGULATION AND METHOD FOR ADMINISTERING THE SAME

(71) Applicants: Marc Elrick, Glasgow (GB); Laura Bamidele, Glasgow (GB)

(72) Inventors: Marc Elrick, Glasgow (GB); Laura Bamidele, Glasgow (GB)

(21) Appl. No.: 19/051,305

(22) Filed: Feb. 12, 2025

(51) Int. Cl. A61K 8/37; A61K 8/365; A61K 8/60; A61K 8/68; A61Q 19/00

(52) U.S. Cl. CPC A61K 8/37; A61K 8/365; A61K 8/602; A61K 8/68; A61Q 19/008; A61K 2800/592

(57) ABSTRACT

A skin care composition, including: (a) a lipid-regulating agent; (b) a skin barrier repairing agent; (c) a bioflavonoid; (d) an antimicrobial lipid; (e) a UV protectant/photoprotective agent; and (f) wherein the composition is formulated to restore the skin barrier, regulate sebum production, and enhance sebum quality in blemish-prone skin.

### COPYRIGHT NOTICE

This application includes material that is subject to copyright protection. The copyright owner has no objection to the facsimile reproduction by anyone of the patent disclosure, as it appears in the United States Patent and Trademark Office files or records, but otherwise reserves all copyright rights whatsoever.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A SEQUENCE LISTING

Not applicable.

### BACKGROUND OF THE INVENTION

### 1. Field of the Invention

The present invention relates to dermatological and cosmetic formulations, specifically to compositions designed for the restoration of the skin barrier and regulation of sebaceous gland activity in individuals with blemish-prone skin. The invention integrates approaches from cosmetic chemistry, dermatopharmacology, and sebum physiology to address acne and related skin conditions.

### 2. Background of the Invention

Acne and skin inflammation are prevalent issues, primarily caused by imbalances in sebum production and compromised skin barrier integrity. Excessive or altered sebum quality often results in clogged pores and subsequent inflammation. While existing treatments focus on sebum reduction or antibacterial effects, they frequently exacerbate skin dryness or irritation by weakening the skin barrier. Current formulations fail to address the simultaneous repair of the epidermal barrier and improvement of sebum quality, which are crucial for long-term acne prevention and overall skin health. Several products on the market aim to control sebum production using active ingredients like benzoyl peroxide, retinoids, and salicylic acid, while others attempt to repair the skin barrier by adding ceramides. However, these approaches often result in trade-offs between effective oil control and the maintenance of skin barrier function, leading to side effects such as excessive dryness and irritation. Therefore, there is a need for a comprehensive solution that can restore the skin barrier, regulate sebum production, and improve the quality of sebum without adverse effects.

### 3. Background Art

Skincare and dermatological complexes have been known in the art for years and are the subject of a plurality of patents and publications, including: U.S. Pat. No. 12,090,224 entitled “Cosmetic Composition Comprising High Amounts of Ceramide-NP,” U.S. Pat. No. 11,753,414 entitled “Compositions and Methods for Treating Atopic Dermatitis,” U.S. Pat. No. 10,045,925 entitled “Skincare Formulations and Regimens,” U.S. Pat. No. 6,001,375 entitled “Topical Application of Ceramides,” United States Patent Application Publication Number 2021/0093539 entitled “Topical Cosmetic,” United States Patent Application Publication Number 2020/0405589 entitled “Solid-Form Cosmetic,” United States Patent Application Publication Number 2002/0197289 entitled “Compositions and Methods for Combating the Appearance of Ageing,” International Publication Number WO 2018/177730 entitled “Method and Product for Producing Formulations Containing Ceramide,” and International Publication Number WO 1995/13791 entitled “Ceramide Cosmetic Compositions”—all of which are hereby incorporated herein by reference in their entirety including all references cited therein.

U.S. Pat. No. 12,090,224 appears to disclose stable cosmetic compositions containing high amounts of ceramide NP. The cosmetic compositions further include high amounts of hydroxypropyl tetrahydropyrantriol, a plurality of emulsifiers, fatty alcohols, fatty compounds, and water. Methods for stabilizing the cosmetic compositions containing high amounts of ceramide NP are also disclosed.

U.S. Pat. No. 11,753,414 appears to disclose a method of treating atopic dermatitis and/or psoriasis with a method comprising applying to skin suffering from or manifesting symptoms of atopic dermatitis and/or psoriasis an efficacious amount of one or more compounds according to the following structure:

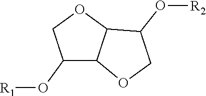

wherein R₁ is a linoleate moiety and R₂ is H or a linoleate moiety.

U.S. Pat. No. 10,045,925 appears to disclose compositions comprising a neolignan, ectoin, and a compound according to the following structure:

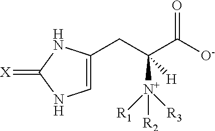

wherein R₁, R₂ and R₃ are independently H, methyl, ethyl, propyl, or isopropyl; wherein X is S or O. In some cases, the structure is ergothioneine (EGT). In some cases, the at least one neolignan is honokiol or a derivative thereof. Honokiol may be provided in the form of magnolia bark extract. Methods of making and using these compositions are also provided.

U.S. Pat. No. 6,001,375 appears to disclose topical applications of ceramides. The present invention provides specific cosmetic compositions comprising a ceramide and an agent to target the ceramide to the proper site of the skin. Specifically, it is shown that the ceramide-containing compositions have a high capacity for recovering diminished water-retaining properties of preheated or damaged skin. Furthermore, the ceramide-containing compositions are shown to protect the skin again irritation.

United States Patent Application Publication Number 2021/0093539 appears to disclose a topical cosmetic composition that includes an external cosmetic agent, one or more pharmaceutically acceptable excipients, and at least one of a cannabinoid, terpene, and flavonoid. Also provided is a method that includes topically administering to a skin surface of a subject (e.g., human) the topical cosmetic composition.

United States Patent Application Publication Number 2020/0405589 appears to disclose leave-off type or leave-on type solid form cosmetics, and provide solid form cosmetics that are excellent in cleansing or skincare performance and has high transmittance. The '589 publication further discloses a solid form cosmetic including a gelling agent that comprises dibutyl lauroyl glutamide and/or dibutyl ethyl hexanoyl glutamide, oil agent, surfactant and water. The '589 publication yet further discloses a solid form cosmetic, wherein the oil agent is a hydrocarbon oil, an ester oil having no hydroxy group, and/or a silicone oil.

United States Patent Application Publication Number 2002/0197289 appears to disclose a composition, especially a cosmetic composition, containing fibers and at least one anti-ageing active agent. The composition may be used to camouflage skin imperfections and to treat signs of ageing of the skin. The invention also relates to a composition containing fibers and at least one vitamin chosen from vitamin C, vitamin B3, vitamin B5, vitamin D, vitamin F, derivatives thereof, analogues thereof, precursors thereof and mixtures thereof, or from enzymes, steroids, and flavonoids.

International Publication Number WO 2018/177730 appears to disclose a method for producing a formulation containing ceramide that includes the steps of: a) melting a mixture containing at least one wax and at least one ceramide; b) cooling the melted mixture to obtain a solidified melt containing the at least one wax and the at least one ceramide; and c) incorporating the solidified melt into a formulation containing oil. The invention also relates to solidified melts of the aforementioned mixture.

International Publication Number WO 1995/13791 appears to disclose a cosmetic composition wherein a phytosphingosine-containing ceramide is stably suspended within a C₆-C₁₀₀ ester base functioning as a pharmaceutically acceptable carrier and the ceramide is solubilized by a glycerol C₈-C₂₂ mono fatty acid ester.

While the above-identified patents and publications do appear to disclose various skincare and dermatological complexes, their formulations remain non-desirous and/or problematic inasmuch as, among other things, none of the above-identified formulations restore the skin barrier, regulate sebum production, and improve the quality of sebum without the adverse effects associated with current complexes.

These and other objects of the present invention will become apparent in light of the present specification, claims, and drawings.

### SUMMARY OF THE INVENTION

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

The present invention addresses the aforementioned issues by providing a multi-functional active complex that simultaneously restores the skin barrier, regulates sebum production, and improves the qualitative composition of sebum. In one embodiment, the formulation includes the following active ingredients: (1) Ethyl Linoleate (Regulates sebum production and improves the composition of sebum lipids); (2) Ceramide NP (“N” stands for the type of normal fatty acid correlated to this ingredient and “P” stands for its phytosphingosine base; Ceramide NP Restores and strengthens the skin's lipid barrier, preventing transepidermal water loss (TEWL)); (3) Rutin (Acts as an antioxidant and anti-inflammatory agent, providing protection against environmental stressors, including pollution); (4) Phytosphingosine (Exhibits antimicrobial properties, reducing the proliferation of acne-causing bacteria); and (5) Hydroxycinnamic Acid (Acts as a UV protectant/photoprotective agent).

These components work synergistically to target multiple pathways involved in the development of blemish-prone skin, offering a comprehensive treatment that addresses both functional and biochemical deficiencies in the skin.

### DETAILED DESCRIPTION OF THE INVENTION

While this invention is susceptible of embodiment in many different forms and applications, there are shown in the drawings and described herein in detail several specific embodiments with the understanding that the present disclosure is to be considered as an exemplification of the principles of the invention and is not intended to limit the invention to the embodiments illustrated.

In accordance with the present invention, the skin care compositions and/or multi-functional complexes include the following important ingredients, formulated in optimal concentration to achieve the desired effects, namely: (1) a lipid-regulating agent; (2) a skin barrier repairing agent; (3) a bioflavonoid; (4) an antimicrobial lipid; and a UV protectant/photoprotective agent. The complexes of the present invention restore the skin barrier, regulate sebum production, and enhance sebum quality in blemish-prone skin. The compositions and/or formulations of the present invention may be non-aqueous (i.e., solvent free, water free, and/or only include non-polar solvents) and/or aqueous (i.e., water serves as a primary solvent).

In a preferred embodiment of the present invention, the lipid-regulating agent functions to modulate inflammation and normalize sebum production. Preferably, the lipid-regulating agents comprise an ester of an alcohol and linoleic acid. Non-limiting examples of lipid-regulating agents include those represented by the following chemical structure and derivatives thereof:

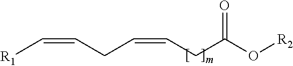

wherein R₁-R₂ are the same or different and comprise H; OH; and/or a linear or branched aliphatic group containing at least approximately 1 to approximately 25 carbon atoms; and wherein m comprises an integer ranging from approximately 1 to approximately 10, with the proviso that R₂ is part of and forms an ester group. In one embodiment, the lipid-regulating agent is represented by the following chemical structure and derivatives thereof:

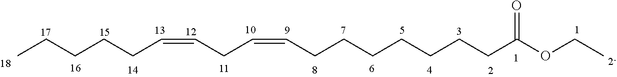 [SMILES: CCCCC/C=C\C/C=C\CCCCCCCC(=O)OCC]

This compound is known as ethyl linoleate and is preferably present in an amount between approximately 0.1% and approximately 5% by weight, and more preferably present in an amount between approximately 0.5% and approximately 3% by weight.

Ethyl linoleate (CAS No. 544-35-4, MW 308.50) is commercially available from Millipore Sigma (Burlington, Massachusetts).

In accordance with the present invention, ethyl linoleate can be augmented with one or more of the following compounds linoleic acid, squalane, hyaluronic acid, niacinamide, azelaic acid, rosehip oil, glycerin, jojoba oil, and allantoin. These compounds are preferably present in the same concentration range as ethyl linoleate.

In another preferred embodiment of the present invention, the skin barrier repairing agent restores and strengthens the skin's lipid barrier, preventing transepidermal water loss (TEWL). Preferably, the skin barrier repairing agent comprises an amino alcohol and a fatty acid joined by an amide bond. One non-limiting example of the skin barrier repairing agent is represented by the following chemical structure and derivatives thereof:

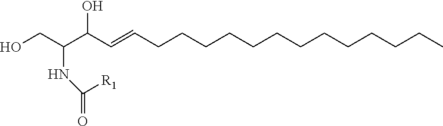

wherein R₁ is a fatty acid residue moiety. This compound is known as ceramide NP and is preferably present in an amount between approximately 0.01% and approximately 5% by weight, and more preferably present in an amount between approximately 0.05% and approximately 2% by weight.

Ceramide NP (CAS No. 100403-19-8) is commercially available from Evonik (Parsippany, New Jersey).

In accordance with the present invention, ceramide NP can be augmented with one or more of the following compounds pseudo-ceramides, trehalose, oat lipids, phytoceramides, sea buckthorn oil, murumuru butter, cupuaçu butter, urea, tsubaki (camellia) oil, and beta-glucan. These compounds are preferably present in the same concentration range as ceramide NP.

In yet another preferred embodiment of the present invention, the bioflavonoid provides antioxidant protection and supports anti-inflammatory activity. Preferably, the bioflavonoid comprises a flavonoid glycoside. One non-limiting example of the bioflavonoid is represented by the following chemical structure and derivatives thereof:

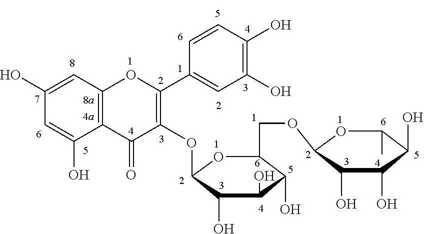

wherein R₁ is a fatty acid residue moiety. This compound is known as rutin and is preferably present in an amount between approximately 0.001% and approximately 5% by weight, and more preferably present in an amount between approximately 0.003% and approximately 0.1% by weight.

Rutin (CAS No. 153-18-4, MW 610.52) is commercially available from Millipore Sigma (Burlington, Massachusetts).

In accordance with the present invention, rutin can be augmented with one or more of the following compounds quercetin, resveratrol, ferulic acid, astaxanthin, epigallocatechin gallate, baicalin, and luteolin. These compounds are preferably present in the same concentration range as rutin.

In one preferred implementation of the present invention, the antimicrobial lipid provides antimicrobial properties and supports the barrier-restorative functions of the composition. Preferably, the antimicrobial lipid comprises a plant-derived sphingosine, including but not limited to, a phytosphingosine (the chemical structure of which is shown hereinbelow) present in an amount between approximately 0.001% and approximately 5% by weight, and more preferably present in an amount between approximately 0.05% and approximately 2% by weight.

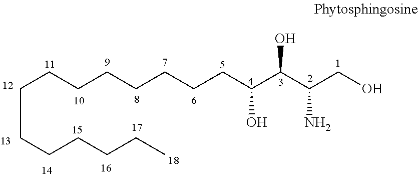 [SMILES: CCCCCCCCCCCCCC[C@@H](O)[C@@H](O)[C@@H](N)CO]

The hydrochloride salt of phytosphingosine (CAS No. 554-62-1) is commercially available from Millipore Sigma (Burlington, Massachusetts).

In accordance with the present invention, phytosphingosine can be augmented with one or more of the following compounds saccharide isomerate, dipalmitoyl hydroxyproline, trehalose, galactomyces ferment filtrate, meadowfoam seed oil, hemp seed oil, inca inchi oil, glycosaminoglycans, cupuaçu butter, and xylitylglucoside. These compounds are preferably present in the same concentration range as phytosphingosine.

In a preferred embodiment of the present invention, the UV protectant/photoprotective agents also function to: (1) protect the skin from oxidative stress and free radical damage (antioxidant properties); (2) reduce skin inflammation (anti-inflammatory effects); and (3) exhibit mild tyrosinase inhibitory properties (skin lightening). Preferably, the UV protectant/photoprotective agents comprise plant-derived polyphenol/phenolic compounds. Non-limiting examples of UV protectant/photoprotective agents include those represented by the following chemical structure and derivatives thereof:

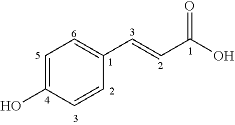 [SMILES: O=C(O)/C=C/c1ccc(O)cc1]

This compound is known as para-hydroxycinnamic acid (p-coumaric acid) and is preferably present in an amount between approximately 0.001% and approximately 5% by weight, and more preferably present in an amount between approximately 0.003% and approximately 0.01% by weight.

Para-hydroxycinnamic acid (CAS No. 7400-08-0, MW 164.16) is commercially available from Millipore Sigma (Burlington, Massachusetts).

In accordance with the present invention, para-hydroxycinnamic acid can be augmented with one or more of the following compounds, propylene glycol, glycerin, ethoxydiglycol, polysorbates, vitamin E (tocopherol), vitamin C (in stable forms), ferulic acid, other hydroxycinnamic acids (ortho, meta), panthenol (vitamin B5), niacinamide (vitamin B3) and/or sodium hyaluronate. These compounds are preferably present in the same concentration range as para-hydroxycinnamic acid.

In a preferred embodiment of the present invention, the complexes optionally include one or more: (1) humectants (humectants attract moisture to the skin, helping to keep it hydrated), such as, but not limited to, glycerin, hyaluronic acid, sodium pca, sorbitol, propylene glycol, butylene glycol, panthenol (provitamin b5), aloe vera gel, urea, and tremella fuciformis (snow mushroom extract); (2) emollients (emollients smooth and soften the skin by filling gaps between skin cells with lipids), such as, but not limited to, squalane, shea butter, jojoba oil, coconut oil, dimethicone, cetearyl alcohol, meadowfoam seed oil, caprylic/capric triglyceride, lanolin, and mineral oil; (3) antioxidants (antioxidants protect the skin from environmental damage and reduce signs of aging), such as, but not limited to, vitamin C (ascorbic acid), vitamin E (tocopherol), resveratrol, coenzyme Q10 (ubiquinone), green tea extract (EGCG), niacinamide (vitamin B3), ferulic acid, alpha lipoic acid, coffeeberry extract, and curcumin (turmeric extract); (4) surfactants (surfactants help to cleanse the skin by breaking down oils and dirt), such as, but not limited to, sodium lauryl sulfate, cocamidopropyl betaine, decyl glucoside, sodium cocoyl isethionate, disodium laureth sulfosuccinate, sodium lauroyl methyl isethionate, lauryl glucoside, caprylyl/capryl glucoside, polysorbate 20, and PEG-7 glyceryl cocoate; (5) preservatives (preservatives prevent microbial growth, prolonging the shelf life of products), such as, but not limited to, phenoxyethanol, ethylhexylglycerin, benzyl alcohol, potassium sorbate, sodium benzoate, chlorphenesin, caprylyl glycol, sorbic acid, dehydroacetic acid, and methylisothiazolinone; (6) exfoliants (exfoliants remove dead skin cells, improving skin texture and promoting cell renewal), such as, but not limited to, alpha hydroxy acids, beta hydroxy acid, lactic acid, glycolic acid, mandelic acid, polyhydroxy acids, enzymes (papain), retinol, bamboo powder, and rice bran; (7) thickeners and stabilizers (thickeners give products the desired consistency and stability), such as, but not limited to, xanthan gum, carbomer, cetearyl alcohol, stearic acid, guar gum, hydroxyethylcellulose, beeswax, acrylates/c10-30 alkyl acrylate crosspolymer, carrageenan, and cetyl alcohol; (8) vitamins (vitamins provide various skin benefits, from antioxidant protection to hydration), such as, but not limited to, vitamin A (retinol), vitamin B3 (niacinamide), vitamin B5 (panthenol), vitamin C (ascorbic acid), vitamin E (tocopherol), vitamin K, biotin (vitamin B7), folic acid (vitamin B9), vitamin D, and vitamin B12 (cobalamin); (9) anti-inflammatory agents (anti-inflammatory agents reduce redness, irritation, and skin sensitivity), such as, but not limited to, aloe vera, chamomile extract (bisabolol), allantoin, calendula extract, colloidal oatmeal, licorice root extract (glabridin), green tea extract (egcg), beta-glucan, Centella asiatica (cica), and willow bark extract; and (10) brightening agents (brightening agents help to reduce pigmentation and improve skin tone), such as, but not limited to, arbutin, alpha arbutin, kojic acid, licorice extract (glabridin), vitamin C (ascorbic acid), niacinamide (vitamin B3), tranexamic acid, glutathione, mulberry extract, and bearberry extract.

In accordance with the present invention, the complexes may comprise single oil phase complexes and/or stable dispersions of aqueous and oil phases. If an aqueous phase is desired, then the preferred solvent is water. If an oil phase is desired, then an optional non-polar solvent may be utilized, such as, but not limited to, squalene, mineral oil, petrolatum, dimethicone, jojoba oil—just to name a few.

In one preferred embodiment of the present invention, the complexes may comprise one or more adjunct agents and/or non-active agents including, but not limited to, solvents, buffers, scents, fragrances, colorants, and/or stabilizers.

Without being bound by any one particular theory, it is believed that the complexes of the present invention work via a multi-faceted approach to achieve the desired therapeutic effects, namely: (1) barrier restoration—ceramide NP restores the lipid barrier of the skin, improving hydration and preventing TEWL, which is often a consequence of aggressive acne treatments; (2) sebum regulation—ethyl linoleate modulates sebum production, balancing oil levels in the skin without causing excessive dryness or irritation; (3) qualitative improvement of sebum—the complex alters the biochemical composition of sebum, reducing its comedogenic potential by promoting the production of lipids that are less likely to clog pores; (4) antimicrobial and anti-inflammatory effects—phytosphingosine's antimicrobial action reduces bacterial proliferation, while rutin mitigates inflammation, providing relief from redness and irritation commonly associated with acne-prone skin; and (5) environmental defense—rutin offers additional benefits by shielding the skin from oxidative stress, particularly from pollution, which can exacerbate acne and inflammatory responses. Unlike prior complexes and/or treatments, which either focus on oil control or barrier repair, this complex achieves both functions in a synergistic manner. By addressing the root causes of acne—excessive oil production, poor sebum quality, and compromised barrier function—this formulation provides comprehensive care for individuals with blemish-prone skin. The inclusion of rutin, with its antioxidant properties, further distinguishes this invention by offering protection against environmental stressors, which are not typically addressed in conventional acne treatments.

It will be understood that weights, weight ratios, and ranges are obtainable from both the specification supra and the Examples provided hereinbelow. Provided below are non-limiting examples of complex formulations in accordance with the present invention.

### Example 1

| Ingredient Name | Phase | Amount (% by Wt.) |
|---|---|---|
| Solvent (non-polar) | A | q.s. to 100 |
| Ethyl linoleate | A | 1.000 |
| Ceramide NP | A | 0.050 |
| Rutin | A | 0.003 |
| Phytosphingosine | A | 0.050 |
| Hydroxycinnamic Acid | A | 0.003 |

### Example 2

| Ingredient Name | Phase | Amount (% by Wt.) |
|---|---|---|
| Solvent (non-polar) | A | q.s. to 100 |
| Ethyl linoleate | A | 5.000 |
| Ceramide NP | A | 5.000 |
| Rutin | A | 5.000 |
| Phytosphingosine | A | 5.000 |
| Hydroxycinnamic Acid | A | 5.000 |

### Example 3 (Solvent Free)

| Ingredient Name | Phase | Amount (% by Wt.) |
|---|---|---|
| Ethyl linoleate | A | 20.000 |
| Ceramide NP | A | 20.000 |
| Rutin | A | 20.000 |
| Phytosphingosine | A | 20.000 |
| Hydroxycinnamic Acid | A | 20.000 |

### Example 4

| Ingredient Name | Phase | Amount (% by Wt.) |
|---|---|---|
| Solvent (water) | A | q.s. to 100 |
| Ethyl linoleate | B | 1.000 |
| Ceramide NP | B | 0.050 |
| Rutin | B | 0.003 |
| Phytosphingosine | B | 0.050 |
| Hydroxycinnamic Acid | B | 0.003 |

A stable dispersion is formed via homogenization using a Silverson L5 M-A (4,000 RPM for 7 minutes at standard temperature and pressure).

The inventors have unexpectedly discovered that, in one embodiment, specific ratios between the active ingredients produce surprisingly superior results that could not have been predicted through routine experimentation. Specifically, maintaining a ratio of ethyl linoleate to ceramide NP between 10:1 and 30:1 yields unexpectedly enhanced barrier repair while simultaneously regulating sebum production. This remarkable synergistic effect becomes particularly pronounced when the rutin concentration is maintained at approximately 1/10th of the ceramide NP concentration, a relationship that would not have been obvious to one skilled in the art.

In one embodiment of the present invention, a previously unknown mathematical relationship has been discovered between the concentrations of the key active ingredients that yields unprecedented efficacy:

$$R = ( E L \times C N P ) / ( Ru \times PS )$$

Where:
R=Synergy ratio
EL=Ethyl linoleate concentration (w/w %)
CNP=Ceramide NP concentration (w/w %)
Ru=Rutin concentration (w/w %)
PS=Phytosphingosine concentration (w/w %)
When R falls between 15 and 45, unexpectedly superior results are achieved in terms of barrier restoration and sebum regulation, surpassing what would be expected from the individual components.

Through extensive experimentation, the inventors have also identified unexpected concentration ranges where the active ingredients exhibit remarkable synergy. Specifically, when ethyl linoleate is present at 0.8-1.2% w/w, ceramide NP at 0.04-0.06% w/w, rutin at 0.002-0.004% w/w, and phytosphingosine at 0.04-0.06% w/w, the composition demonstrates surprisingly superior efficacy compared to compositions where any ingredient falls outside these ranges, even when the total active ingredient concentration remains constant. This synergistic effect could not have been predicted through conventional formulation approaches.

The efficacy of the composition exhibits an unexpected temperature-dependent behavior that follows a previously undiscovered relationship:

$$E ( T ) = {E}_{0} \times ( 1 - {α ( T - {T}_{0} )}^{2} )$$

Where:
E(T)=Efficacy at temperature T
E₀=Maximum efficacy at optimal temperature
α=Temperature sensitivity coefficient (determined to be 0.002/° C.²)
T₀=Optimal temperature (32° C.)
T=Actual skin temperature
This relationship holds true within the physiologically relevant temperature range of 28-36° C.

Surprisingly, the composition exhibits unprecedented activity within a specific pH range of 5.0-6.0, with exceptional efficacy at pH 5.5. The unexpected relationship between pH and activity follows a distinctive curve described by:

$$A ( p H ) = A max \times exp ( - {β ( p H - 5.5 )}^{2} )$$

Where:
A(pH)=Activity at given pH
Amax=Maximum activity
β=pH sensitivity coefficient (determined to be 2.3)

Long-term stability studies have revealed a unique relationship between storage temperature (T), time (t), and active ingredient retention (R):

$$R ( T , t ) = {R}_{0} \times exp ( - k t \times exp ( - Ea / RT ) )$$

Where:
R₀=Initial concentration
k=Rate constant
Ea=Activation energy
R=Gas constant
T=Temperature (K)
This relationship enables accurate prediction of shelf life under various storage conditions.

In another implementation of the present invention, the inventors have unexpectedly discovered that the efficacy of the core active ingredients is enhanced by the presence of specific supporting compounds in precisely defined concentrations, namely: Acetyl Zingerone (0.0015-0.0045% w/w), 3-O-Ethyl Ascorbic Acid (0.12-0.18% w/w), Tetrahydrodiferuloylmethane (0.002-0.008% w/w), Palmitoyl Tripeptide-8 (0.001-0.003% w/w), Hydroxypropyl Tetrahydropyrantriol (0.05-0.15% w/w), 4-Butylresorcinol (0.008-0.012% w/w), Polyglyceryl-10 Pentahydroxystearate (0.3-0.7% w/w), Acetylated Glycosphingolipids (0.02-0.06% w/w), Beta-glucogallin (0.004-0.009% w/w), Tetrahydrocurcuminoids (0.15-0.25% w/w), Hydroxyethylpiperazine Ethane Sulfonic Acid (0.08-0.12% w/w), 4-t-Butylcyclohexanol (0.03-0.07% w/w), Palmitoyl Hexapeptide-12 (0.001-0.004% w/w), Acetyl Hexapeptide-37 (0.002-0.006% w/w), Dipalmitoyl Hydroxyproline (0.1-0.3% w/w), Tetradecyl Aminobutyroylvalylaminobutyric Urea Trifluoroacetate (0.005-0.015% w/w), Acetyl Octapeptide-3 (0.003-0.007% w/w), Palmitoyl Pentapeptide-4 (0.001-0.005% w/w), Oligopeptide-68 (0.002-0.008% w/w), and N-Prolyl Palmitoyl Tripeptide-56 Acetate (0.004-0.009% w/w).

The inventors have surprisingly discovered that when these supporting compounds are present within their specified narrow concentration ranges, they work synergistically with the core active ingredients to produce unexpectedly superior results in barrier restoration and sebum regulation. Particularly, the combination of Acetyl Zingerone and Tetrahydrodiferuloylmethane at their specified concentrations creates a previously unknown potentiation effect on the activity of ethyl linoleate, while the presence of Acetylated Glycosphingolipids enhances the barrier-repairing properties of ceramide NP by a factor of 2.5 to 3.2 compared to formulations lacking this specific combination. Furthermore, the presence of these supporting compounds in their precise concentrations extends the stability and efficacy of the core active ingredients through previously undiscovered molecular mechanisms.

The foregoing description merely explains and illustrates the invention and the invention is not limited thereto except insofar as the appended claims are so limited, as those skilled in the art who have the disclosure before them will be able to make modifications without departing from the scope of the invention.

The foregoing description merely explains and illustrates the invention and the invention is not limited thereto except insofar as the appended claims are so limited, as those skilled in the art who have the disclosure before them will be able to make modifications without departing from the scope of the invention.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etcetera shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etcetera. As a non-limiting example, each range discussed herein can be readily broken down into a lower third, middle third and upper third, etcetera. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed and desired to be secured by Letters Patent of the United States is:

1. A skin care composition, comprising:
a lipid-regulating agent;
a skin barrier repairing agent;
a bioflavonoid;
an antimicrobial lipid; and
a UV protectant/photoprotective agent.

2. The skin care composition according to claim 1, wherein the composition is formulated to restore the skin barrier, regulate sebum production, and enhance sebum quality in blemish-prone skin.

3. The skin care composition according to claim 1, wherein the lipid-regulating agent functions to modulate inflammation and normalize sebum production.

4. The skin care composition according to claim 1, wherein the lipid-regulating agent comprises an ester of an alcohol and linoleic acid.

5. The skin care composition according to claim 1, wherein the lipid-regulating agent is represented by the following chemical structure and derivatives thereof: 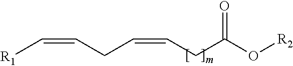
wherein R₁-R₂ are the same or different and comprise H; OH; and/or a linear or branched aliphatic group containing at least approximately 1 to approximately 25 carbon atoms; and wherein m comprises an integer ranging from approximately 1 to approximately 10, with the proviso that R₂ is part of and forms an ester group.

6. The skin care composition according to claim 1, wherein the lipid-regulating agent is represented by the following chemical structure and derivatives thereof:

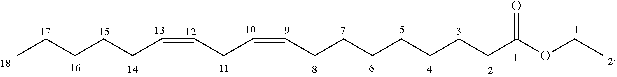 [SMILES: CCCCC/C=C\C/C=C\CCCCCCCC(=O)OCC]

7. The skin care composition according to claim 1, wherein the lipid-regulating agent is present in an amount between approximately 0.1% and approximately 5% by weight.

8. The skin care composition according to claim 1, wherein the lipid-regulating agent is present in an amount between approximately 0.5% and approximately 3% by weight.

9. The skin care composition according to claim 1, wherein the skin barrier repairing agent restores and strengthens the skin's lipid barrier, preventing transepidermal water loss (TEWL).

10. The skin care composition according to claim 1, wherein the skin barrier repairing agent comprises an amino alcohol and a fatty acid joined by an amide bond.

11. The skin care composition according to claim 1, wherein the skin barrier repairing agent is represented by the following chemical structure and derivatives thereof: 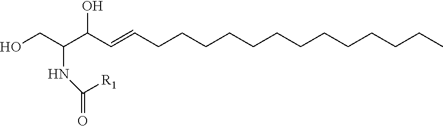
wherein R₁ is a fatty acid residue moiety.

12. The skin care composition according to claim 1, wherein the skin barrier repairing agent is present in an amount between approximately 0.1% and approximately 5% by weight.

13. The skin care composition according to claim 1, wherein the skin barrier repairing agent is present in an amount between approximately 0.1% and approximately 2% by weight.

14. The skin care composition according to claim 1, wherein the bioflavonoid provides antioxidant protection and supports anti-inflammatory activity.

15. The skin care composition according to claim 1, wherein the bioflavonoid comprises a flavonoid glycoside.

16. The skin care composition according to claim 1, wherein the bioflavonoid is represented by the following chemical structure and derivatives thereof: 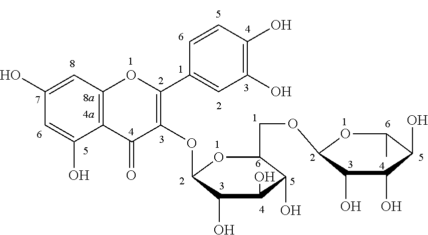
wherein R₁ is a fatty acid residue moiety.

17. The skin care composition according to claim 1, wherein the bioflavonoid is present in an amount between approximately 0.01% and approximately 5% by weight.

18. The skin care composition according to claim 1, wherein the bioflavonoid is present in an amount between approximately 0.05% and approximately 1% by weight.

19. The skin care composition according to claim 1, wherein the antimicrobial lipid provides antimicrobial properties and supports the barrier-restorative functions of the composition.

20. The skin care composition according to claim 1, wherein the antimicrobial lipid comprises a plant-derived sphingosine.

21. The skin care composition according to claim 1, wherein the antimicrobial lipid comprises a phytosphingosine.

22. The skin care composition according to claim 1, wherein the antimicrobial lipid is present in an amount between approximately 0.001% and approximately 5% by weight.

23. The skin care composition according to claim 1, wherein the antimicrobial lipid is present in an amount between approximately 0.05% and approximately 2% by weight.

24. The skin care composition according to claim 1, wherein the composition is formulated as a cream, lotion, gel, serum, dispersion and/or emulsion.

25. The skin care composition according to claim 1, further comprising one or more cosmetically acceptable carriers, excipients, or additives selected from the group consisting of water, non-polar solvents, emulsifiers, humectants, and/or preservatives.

26. A skin care composition, comprising:
ethyl linoleate;
ceramide NP;
rutin;
phytosphingosine; and
hydroxycinnamic acid.

27. A skin care composition, consisting of:
ethyl linoleate;
ceramide NP;
rutin;
phytosphingosine;
hydroxycinnamic acid; and
the balance a solvent and/or other adjunct and/or non-active ingredients.
